(19) United States
(12) Patent Application Publication
(10) Pub. No.: US 2005/0181082 A1
(43) Pub. Date: Aug. 18, 2005

(54) Morinda citrifolla based antifungal formulations and methods

(76) Inventors: Fumiyuki Isami, Tokyo (JP); John J. Wadsworth, Orem, UT (US); Scott Gerson, Brewster, NY (US); 'Afa K. Palu, Orem, UT (US); Bing-Nan Zhou, Pleasant Grove, UT (US); Chen Su, West Jordan, UT (US); Claude Jarakae Jensen, Cedar Hill, UT (US); Stephen P. Story, Alpine, UT (US)

Correspondence Address: Michael F. Krieger;Kirton & McConkie, P.O. Box 45120, Salt Lake City, UT, 84145, US

(21) Appl. No.: 11/091,051

(22) Filed: Mar. 28, 2005

Related U.S. Application Data
(63) Continuation of application No. 10/439,596, filed on May 16, 2003.
(60) Provisional application No. 60/382,246, filed on May 21, 2002.

(51) Int. Cl. A61K 35/78

(52) U.S. Cl. 424/769; 424/777

(57) ABSTRACT

The present invention provides a formulation which may be utilized in agricultural practice that is eco-friendly and effective as plant growth promotion agent, soil improvement agent, bactericide and insecticide agent, disease and harmful insect prevention agent, and is suitable for organic farming. The formulation of the present invention is comprised of a Morinda citrifolia product or extract. The formulation of the present invention may be applied to fruit vegetables, leafy vegetables, root vegetables, grains as well as flowers and shrubs, increasing the amount of yield and extending freshness period after harvest. Further, the present invention relates to antifungal and antibacterial activity of processed Morinda citrifolia products, as well as from various fractions of extracts from these processed products and the Morinda citrifolia L. plant, and related methods to determine mean inhibitory concentrations. In particular, the present invention relates to ethanol, methanol and ethyl acetate extracts from Morinda citrifolia L. and their inhibitory activities on common fungi and bacteria and the identification of mean inhibitory concentrations.

### RELATED APPLICATIONS

This application is a continuation-in-part of U.S. patent application Ser. No. 10/439,596, filed May 16, 2003, entitled, “Antifungal Effects of Morinda Citrifolia,” which claims priority to U.S. Provisional Application Ser. No. 60/382,246, filed May 21, 2002, entitled, “Antifungal Activity and Mean Inhibitory Concentration of Selected Extracts from Morinda citrifolia L. and related methods.”

### BACKGROUND

1. Field of the Invention The present invention relates to Morinda citrifolia based composition, which may be utilized agriculturally to reduce fungal infections increase crop yields, and help maintain the freshness of the crop after harvest.

2. Background of the Invention and Related Art

Organic refers to agricultural production systems used to produce food and fiber. Various agricultural products are produced organically, including produce, grains, meat, dairy, eggs, and fibers such as cotton, flowers, and processed food products. Organic farming management relies on the use of natural mechanism to disrupt habitat for pest organisms, and the purposeful maintenance and replenishment of soil fertility. Organic farmers do not utilize synthetic pesticides or fertilizers. Organic growers do not utilize synthetic agrochemicals, irradiation and genetically engineered foods or ingredients. To maintain the integrity of food without artificial ingredients or preservatives organic foods are processed as little as possible. Because organic farmers adhere to these practices, organic food is far less likely to contain pesticide residues than conventional food. Baker, B. P., et al., Pesticide residues in conventional, integrated pest management (IPM)-grown and organic food: insights from three US data sets, 19 FOOD ADDITIVES AND CONTAMINANTS 427-446 (2002)(13% of organic produce samples vs. 71% of conventional produce samples contained a pesticide residue, when long-banned persistent pesticides were excluded).

The organic food market is large and growing. Approximately 2% of the U.S. food supply is grown using organic methods. Over the past decade, sales of organic products have shown an annual increase of at least 20%, the fastest growing sector of agriculture. In 2001, retail sales of organic food were projected to be $9.3 billion (Organic Consumer Trends 2001. Published by the Natural Marketing Institute, in partnership with the Organic Trade Association, http://www.ota.com/consumer_trends_{—}2001.htm). The international market for organic foods is also growing. In particular Japan and Germany are becoming important international organic food markets.

The cost of organic food is higher than that of conventional food, because organic farmers substitute labor and intensive management for chemicals. In doing so organic farmers absorb some cost previously external to conventional farming practices (e.g., health and environmental costs). Some of the costs associated with organic farming include cleanup of polluted water and remediation of pesticide contamination. Additionally, prices for organic foods include costs of growing, harvesting, transportation and storage. In the case of processed foods, processing and packaging costs are also included.

In addition to higher cost, organic farming typically yields fewer crops than conventional farming techniques. Based on 154 growing seasons' worth of data on various crops, organic crops yielded 95% of crops grown under conventional, high-input conditions.

Organic farmers build healthy soils by nourishing the living component of the soil, the microbial inhabitants that release, transform, and transfer nutrients. Soil organic matter contributes to good soil structure and water-holding capacity. Organic farmers feed soil biota and build soil structure and water-holding capacity. Organic farmers feed soil biota and build soil organic matter with cover crops, compost, and biologically based soil amendments. These produce healthy plants that are better able to resist disease. As a last resort, certain botanical or other non-synthetic pesticides may be applied.

Conventional and organic farmer face the difficult task of ameliorating unwanted microorganism that decrease yield and quality of food products. To avoid utilizing synthetic amendments during the growing process organic farmers particularly must depend on biologically based treatments. Despite the existence of tens of thousands of antimicrobial compounds, the ability of microorganisms to develop resistance to even the most recent and powerful antimicrobial compounds or treatments is rapid. In order to keep pace with the increasing need for new antimicrobials, it is important that new compounds be discovered. Some of these may even come from unexpected sources (see e.g., the development of penicillin).

Juice from Morinda citrifolia is known to have many useful properties and contain many nutritious elements. Herbs, health foods, pet foods, cosmetics and other products have been developed utilizing some of the elements of the fruit. However, an agricultural composition utilizing various products from Morinda citrifolia is not yet known.

Thus, organic and conventional farming practice may be improved by increasing yields, increasing the quality of food products produced and by decreasing the costs of organic farming. The present invention provides relates to compositions and methods that can be utilized by both conventional and organic farmers to increases yields and the quality of food produced.

### SUMMARY OF THE INVENTION

The present invention aims to provide Morinda citrifolia based compositions for agricultural use, which are effective but do not have a deleterious effect on ecological systems and are suitable for organic farming. Implementation of the present invention takes place in association with the utilization of juice, puree, and other extracts or parts from the plant known as Morinda citrifolia L. Embodiments of the invention include compositions designed for agricultural use, wherein the particular composition include a fertilizer, growth promotion agent for crops, soil improvement agent, anti-bacteria and insecticide agent, an antimicrobial, and disease and harmful insect prevention agent. Moreover, the agricultural composition is comprised of natural materials having such effects as promotion of crop growth, improvement in crop quality, improvement in resistance against disease and harmful insects, increase in the amount of crop yield, enhancement in sugar and taste, and improvement in freshness after harvest.

The present invention provides compositions for agricultural use, comprising various elements from Morinda citrifolia in isolation or in combination with other ingredients. The present invention provides various Morinda citrifolia based compositions, which may be comprised of extracts or processed products derived from the fruit, leaves, stem, seed bark and/or root of Morinda citrifolia. The invention also provides for the combination of various elements from Morinda citrifolia with additional ingredients to enhance the agricultural utility of the described compositions. For example, one embodiment of the present invention discloses utilizing extracts from Morinda citrifolia fruit, leaves, stem, seed and/or root, which have been diluted by a factor of 1-10,000 times (by weight) with water. The compositions of the present invention possess the ability to increase amount of crop yields and maintain freshness of the crop after harvesting.

Further, the present invention relates to antifungal and antibacterial activity of extracts from Morinda citrifolia L. and related methods to determine mean inhibitory concentrations. In particular, the present invention relates to ethanol, methanol and ethyl acetate extracts from Morinda citrifolia L. and their inhibitory activities on common fungi and bacteria and the identification of mean inhibitory concentrations.

In accordance with the invention as embodied and broadly described herein, the present invention features various methods for inhibiting, preventing, and destroying existing harmful fungi and microbial activity and growth using active compounds and/or ingredients extracted from and existing within one or more processed Morinda citrifolia products. The Morinda citrifolia products are preferably supplied in a formulation designed to effect the inhibition of undesirable microbial activity.

The processed Morinda citrifolia product may comprise a variety of types, including, but not limited to, processed Morinda citrifolia fruit juice, processed Morinda citrifolia puree juice, processed Morinda citrifolia dietary fiber, processed Morinda citrifolia oil, processed Morinda citrifolia fruit juice concentrate, processed Morinda citrifolia puree juice concentrate, and processed Morinda citrifolia oil extract.

The present invention also features a formulation for inhibiting and treating fungi and microbial activity and growth, wherein the formulation comprises at least one or more processed Morinda citrifolia products. Within the processed Morinda citrifolia products are Morinda citrifolia fractions or extracts that specifically exhibit antifungal and antimicrobial activities. The formulation also may comprise other natural ingredients.

### DETAILED DESCRIPTION OF THE PREFERRED EMBODIMENTS

The agricultural formulations and methods of the present invention may be produced by extracting effective components from fruit, leaves, stem, seeds and/or root of Morinda citrifolia. Additionally, the present invention relates to methods for determining the activity and mean inhibitory concentration of extracts of Morinda citrifolia L. against common fungi and bacteria. In particular, the present invention relates to ethanol, methanol and ethyl acetate extracts and various fractions from Morinda citrifolia L. and the antifungal and antibacterial effect of these in regards to their determined mean inhibitory concentrations and mean lethal concentrations as existing within a formulation, which concentrations are based upon various experimental studies.

The compositions and formulations of the present invention, as generally described herein, may be designed to comprise variations. Thus, the following more detailed description of the embodiments of the formulations and methods of the present invention is not intended to limit the scope of the invention, as claimed, but is merely representative of the presently preferred embodiments of the invention.

In the disclosure and in the claims the singular forms “a,” “an,” and “the” include plural referents unless the context clearly dictates otherwise.

In describing and claiming the present disclosure, the following terminology will be used in accordance with the definitions set out below. As used herein, the terms “comprising,” “including,” “containing,” “characterized by,” and grammatical equivalents thereof are inclusive or open—ended terms that do not exclude additional, unrecited elements or method steps. As used herein, the phrase “consisting of” and grammatical equivalents thereof exclude any element, step, or ingredient not specified in the claim. As used herein, an “effective amount” is an amount sufficient to effect beneficial or desired results. An effective amount can be administered in one or more administrations, applications or treatments. For example, an effective amount of a Morinda citrifolia based composition is an amount sufficient to provide antimicrobial activity, and ameliorate related conditions. Such effective amounts can be determined without undue experimentation by those skilled in the art.

The following disclosure of the present invention is grouped into three subheadings, namely “General Discussion of Morinda citrifolia and the Methods Used to Produce Processed Morinda citrifolia Products,” “Agricultural Formulations and Methods of Administration” and “Antimicrobial Activity.” The utilization of the subheadings is for convenience of the reader only and is not to be construed as limiting in any sense.

1. General Discussion of Morinda citrifolia and the Methods Used to Produce Processed Morinda citrifolia Products

The Indian Mulberry or Noni plant, known scientifically as Morinda citrifolia L. (Morinda citrifolia ), is a shrub or small tree. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in other products. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 120 days. Most fruit containers are held for 7 to 14 days before processing. The containers can optionally be stored under refrigerated conditions or ambient/room temperature conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers):
acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid. The present invention contemplates utilizing all parts of the M. citrifolia plant alone, in combination with each other or in combination with other ingredients. The above listed portions of the M. citrifolia plant are not an exhaustive list of parts of the plant to be used but are merely exemplary. Thus, while some of the parts of the M. citrifolia plant are not mentioned above (e.g., seed from the fruit, the pericarp of the fruit, the bark or the plant) the present invention contemplates the use of all of the parts of the plant.

In order to obtain extract from leaves, stem, seeds and/or roots of Morinda citrifolia, first these raw materials are chopped. Next, an extraction method is utilized to isolate ingredients of interest. In a preferred embodiment of the invention a hot water extraction method is utilized, wherein water, five to ten times in amount, is added and heated at the temperature of 95° C. or an extraction method wherein organic solvent such as ethanol, methanol, hexane and the like or mixture of water and organic solvent are used may be applied. Moreover, wet pressure and heat process using ordinary autoclave equipment may be applied. Furthermore, treatment processes using cellulose hydrolysis enzyme may be added to aforementioned processes. After removing insoluble components through filtering, if desired, from extract obtained from leaves, stems, seeds and/or roots, organic solvent is removed and extract of the present invention is obtained. This extract may be pasteurized, if necessary, or concentrated or dried. Drying may be achieved using ordinary spray drying or freeze drying. The extract may be stored under cooling or freezing conditions.

Moreover, oil may be extracted from seeds. Oil may be obtained by drying, crushing, and squeezing seeds with a press. More oil may be extracted from seed cake residue by adding hexane solution and the like. The oil contains fatty acid such as linoleic acid, oleic acid, palmitic acid and stearic acid in the form of triglycerides.

Recently, as mentioned, many health benefits have been discovered stemming from the use of products containing Morinda citrifolia. One benefit of Morinda citrifolia is found in its ability to isolate and produce Xeronine. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase, which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase.

Xeronine protects and keeps the shape and suppleness of protein molecules so that they may be able to pass through the cell walls and be used to form healthy tissue. Without these nutrients going into the cell, the cell cannot perform its job efficiently. Xeronine assists in enlarging the membrane pores of the cells. This enlargement allows for larger chains of peptides (amino acids or proteins) to be admitted into the cell. If these chains are not used they become waste. Additionally, Xeronine, which is made from Proxeronine, assists in enlarging the pores to allow better absorption of nutrients. Because of its many benefits, Morinda citrifolia has been known to provide a number of anecdotal effects

Favorably, this invention provides a method of treating and inhibiting fungal and other microbial activity or growth with a Morinda citrifolia-based formulation without any significant tendency to cause deleterious environmental effects.

As used herein, the term Morinda citrifolia juice refers to a product that includes juice processed from the fruit of the Indian Mulberry or Morinda citrifolia L. plant. In one embodiment, Morinda citrifolia juice includes reconstituted fruit juice from pure juice puree of French Polynesia. The composition or formulation comprising at least one processed Morinda citrifolia product may also include other ingredients. In a further embodiment, Morinda citrifolia juice is not processed from dried or powdered Morinda citrifolia.

2. Formulations and Methods of Administration

The following section details some preferred embodiments of Morinda citrifolia-based formulations and methods of utilizes said formulations in an agricultural setting to improve the yield and quality of food produced, particularly by inhibiting and preventing deleterious microbial growth and by providing additional nutrients to the developing plants.

The present invention advances fungal and other antimicrobial inhibitors by providing a composition formulated with one or more processed Morinda citrifolia products derived from the Indian Mulberry plant. The Morinda citrifolia is incorporated into various carriers or compositions suitable for agricultural use.

Agricultural formulations of the present invention may be produced by forming extract or mixture of extract from fruit, stem, seed and/or root of Morinda citrifolia obtained using aforementioned procedures made into liquid, granule, powder or paste agent with appropriate carrier materials. The agricultural formulations of the present invention may be used by dissolving or dispersing in water. Moreover, the formulations of the present invention may be mixed with a fertilizer component such as ammonium sulfate, urea, potassium, nitrogen and ammonium chloride, various composts, various manures, chicken manure, cow manure, guano, worm castings, insect manure, saw dust, rice bran, garlic oil, fish oil, vermiculite, montmorillonite, active carbon, charcoal, diatomite, talc, alfalfa meal and pellets, nitrogen, phosphorus, potassium, dried shredded remains of sugar beets, corn gluten, cottonseed meal, extracts or pulverized parts of several kelp or algae, soybean meal, animal processing by-products, blood meal, bonemeal, and fish by products.

Agricultural activation agent of the present invention may be applied to fruits vegetables, leafy vegetables, root vegetables, grains, and flower and bulbs. In fact, the following usage may be suggested: the formulation may be sprayed or irrigated in the soil prior to planting or during plant growth; coat or disperse the plant during cutting, dividing or re-planting the plant; coat or disperse seed or bulb during planting; coat or disperse wilting flowers and shrubs; disperse water grown plant; coat or disperse plants infected with bacteria or virus; coat or disperse cut flowers after harvest; coat or disperse crop and flower after harvest.

In one exemplary embodiment, the composition of the present invention comprises one or more of a processed Morinda citrifolia (e.g. Morinda citrifolia fruit juice or fruit juice or puree juice) product present in an amount by weight between about 0.01 and 100 percent by weight, and preferably between 0.01 and 95 percent by weight. Several embodiment of formulations are provided below. However, these are only intended to be exemplary as one ordinarily skilled in the art will recognize other formulations or compositions comprising the processed Morinda citrifolia product.

The processed Morinda citrifolia product comprises at least one of the active ingredient, such as Quercetin and Rutin, and others, for effectuating the inhibition of fungal activity.

Active ingredients within the processed Morinda citrifolia product may be extracted out using various alcohol or alcohol-based solutions, such as methanol, ethanol, and ethyl acetate, and other alcohol-based derivatives using procedures and processes commonly known in the art. The active ingredients of Quercetin and Rutin are present in amounts by weight ranging from 0.01-10 percent of the total formulation or composition. If desired, these amounts may be concentrated into a more potent concentration in which they are present in amounts ranging from 10 to 100 percent.

In one exemplary embodiment, the method comprises the steps of (a) formulating a composition comprising in part a processed Morinda citrifolia product present in an amount between about 0.01 and 95 percent by weight, wherein the composition also comprises a carrier, such as water or purified water, and may also comprise other natural or artificial ingredients including selected fertilizers; (b) administering the composition into the soil or plant, such that the processed Morinda citrifolia product is allowed to be incorporated or come into contact with a plant; (c) repeating the above steps as often as necessary to provide an effective amount of the processed Morinda citrifolia product needed to inhibit and/or prevent fungal and other microbial activity or growth, while simultaneously increasing crop yield. One ordinarily skilled in the art will recognize that the amount of composition and frequency of use may vary from one agricultural situation to another.

The following tables illustrate or represent some of the preferred formulations or compositions contemplated by the present invention. As stated, these are only intended as exemplary embodiments and are not to be construed as limiting in any way.

| | |
|---|---|
| Ingredients | Percent by Weight |

Formulation One

| | |
|---|---|
| Morinda citrifolia puree juice or fruit juice | 100% |

Formulation Two

| | |
|---|---|
| Morinda citrifolia fruit juice | 85-99.99% |
| Water | 0.01-15% |

Formulation Three

| | |
|---|---|
| Morinda citrifolia fruit juice | 0.01-15% |
| Water | 85-99.99% |

Formulation Four

| | |
|---|---|
| Morinda citrifolia fruit juice | 15-85% |
| Water | 15-85% |

Formulation Five

| | |
|---|---|
| Morinda citrifolia fruit juice | 20-90.8% |
| water | 0.1-50% |
| Fertilizer | 0.1-30% |

Formulation Six

| | |
|---|---|
| Morinda citrifolia fruit juice | 0.1-30% |
| water | 0.1-50% |
| Fertilizer | 20-90.8% |

Formulation Seven

| | |
|---|---|
| Extracted Ingredient from Morinda citrifolia fruit, | 100% |
| pericarp stem, seed and/or root | |

Formulation Eight

| | |
|---|---|
| Extracted Ingredient from Morinda citrifolia fruit, | 85-99.99% |
| pericarp stem, seed and/or root | |
| water | 0.01-15% |

Formulation Nine

| | |
|---|---|
| Extracted Ingredient from Morinda citrifolia fruit, | 0.01-15% |
| pericarp stem, seed and/or root | |
| water | 85-99.99% |

Formulation Ten

| | |
|---|---|
| Extracted Ingredient from Morinda citrifolia fruit, | 50-90.98% |
| pericarp stem, seed and/or root | |
| water | 0.01-50% |
| Fertilizer | 0.01-30% |

Formulation Eleven

| | |
|---|---|
| Extracted Ingredient from Morinda citrifolia fruit, | 0.1-30% |
| pericarp stem, seed and/or root | |
| water | 1-99.9% |
| Fertilizer | 1-99.9% |

Formulation Twelve

| | |
|---|---|
| Morinda citrifolia oil | 0.1-30% |
| carrier medium | 70-99.9% |
| other ingredients (e.g., Fertilizer) | 1-95% |

Formulation Thirteen

| | |
|---|---|
| Morinda citrifolia product | 10-80% |
| carrier medium | 20-90% |

Formulation Fourteen

| | |
|---|---|
| Morinda citrifolia product | 5-80% |
| carrier medium | 20-95% |

Formulation Fifteen

| | |
|---|---|
| Morinda citrifolia oil or oil extract | 0.1-20% |
| carrier medium | 20-90% |

Formulation Sixteen

| | |
|---|---|
| Morinda citrifolia puree juice or fruit Juice | 0.1-80% |
| Morinda citrifolia oil | 0.1-20% |
| carrier medium | 20-90% |

Formulation Seventeen

| | |
|---|---|
| Morinda citrifolia puree juice concentrate or fruit | 100% |
| juice concentrate | |

Formulation Eighteen

| | |
|---|---|
| Morinda citrifolia fruit juice concentrate or puree | 85-99.99% |
| juice concentrate | |
| Water | 0.1-15% |

Formulation Nineteen

| | |
|---|---|
| Morinda citrifolia puree juice or fruit juice fraction | 100% |

Formulation Twenty

| | |
|---|---|
| Morinda citrifolia fruit juice fraction | 85-99.99% |
| Water | 0.1-15% |

Formulation Twenty One

| | |
|---|---|
| Morinda citrifolia fruit juice fraction | 85-99.99% |
| Fertilizer | 0.1-15% |

Formulation Twenty Two

| | |
|---|---|
| Morinda citrifolia fruit juice fraction | 50-90% |
| water | 0.1-50% |
| Fertilizer | 0.1-30% |

Formulation Twenty Three

| | |
|---|---|
| Morinda citrifolia puree juice fraction | 85-99.9% |
| water | 0.1-15% |

Formulation Twenty Four

| | |
|---|---|
| Morinda citrifolia juice | 0.1-80% |
| Extracted ingredient(s) from Morinda citrifolia | 0.1-20% |
| Fertilizer | 20-90% |

In one example, which is not meant to be limiting in any way, the beneficial Morinda citrifolia is processed into TAHITIAN NONI ® juice manufactured by Morinda, Incorporated of Orem, Utah.

In an exemplary embodiment, formulation comprises the ingredients of: a processed Morinda citrifolia product present in an amount by weight between about 10-80 percent; and a carrier medium present in an amount by weight between about 20-90 percent.

In this embodiment, the processed Morinda citrifolia product may comprise one or more of a processed Morinda citrifolia fruit juice, processed Morinda citrifolia puree juice, processed Morinda citrifolia fruit or puree juice concentrate, extracted ingredient(s) from Morinda citrifolia, and/or processed Morinda citrifolia oil extract product.

In another exemplary embodiment, the formulation comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent.

The carrier medium identified in the above-identified Formulations may comprise any ingredient capable of being introduced into or onto the tissues of a plant, and that is also capable of providing the carrying medium to the processed Morinda citrifolia product. Specific carrier mediums formulations are well known in the art and not described in detail herein. The purpose of the carrier medium is as stated, to provide a means to embody the processed Morinda citrifolia product within the forumlation that is capable of being introduced into or onto the tissues of a plant.

3. Antimicrobial Activity

The following examples set forth and present the preventative and treatment effects of the processed Morinda citrifolia products on fungal activity. These examples are not intended to be limiting in any way, but are merely illustrative of the benefits and advantageous, as well as the remedial effects, of the Morinda citrifolia products.

### EXAMPLE ONE

A study was conducted to determine the mean inhibitory concentrations of certain extracts from Morinda citrifolia against activity of common fungi and bacteria. In this study an attempt has been made to identify antimicrobial activity from Morinda citrifolia using a “top down” approach. A reproducible assay was developed, and initial studies have indicated that an antimicrobial component from Morinda citrifolia can be extracted. The study demonstrated that ethanol, methanol and ethyl acetate extracts of Morinda citrifolia were found to exhibit antimicrobial activity when tested against S. aureus, E. coli, C. albicans, T. mentagrophytes and A. niger.

In recent years, in an attempt to discover new antimicrobial compounds, many different sources have been explored. In this study a Mean Inhibitory Concentration (MIC) protocol was developed and then used to test ethanol, methanol, and ethyl acetate extracts of Morinda citrifolia, for antifungal and antimicrobial activity against Aspergillus niger (ATCC 6275); Candida albicans (ATCC 10231); Trichophyton mentagrophytes (ATCC 9533); Staphlococcus aureus (ATCC 29213); and Escherichia coli (ATCC 25922).

Liquid extracts were obtained, and tested in microliter wells in duplicate. Quantities of the extracts, ranging from 6 ul to 200 μl, were placed in wells and dried. A McFarland 0.5 solution of each organism was prepared, and a 1/100 suspension into the appropriate media was made. This organism suspension was added to each well, and incubated for an appropriate amount of time at the appropriate temperature. Plates were then examined for growth, and MIC's were determined. All duplicate results agreed within one dilution. The ethyl acetate extracts had the least amount of antimicrobial activity, only showing activity when tested against T. mentagrophytes and S. aureus. The ethanol extracts showed antimicrobial activity against all of the organisms tested. This activity ranged from off-scale on the low end when tested against T. mentagrophytes, to high on-scale results for A. niger. Methanol extracts also had activity against all of the organisms tested, and ranged from off-scale on the low end when tested against T mentagrophytes, to high on-scale results for A. niger. These results indicate that at least some extracts of Morinda citrifolia contain antimicrobial activity. A more detailed description of this test follows.

The materials used in this test included several cultured microorganisms, namely, S. aureus ATCC 29213, E. coli ATCC 25922, C. albicans ATCC 10231, T. mentagrophytes ATCC 9533 and A. niger ATCC 6275. Initial cultures were developed as per the manufacturer's instructions. Prior to testing, S. aureus and E. coli were plated on Trypticase Soy Agar Plates, and incubated for 18-24 hours at 37° C. C. albicans, T. mentagrophytes and A. niger were plated on Saboraud Dextrose Agar plates, and incubated for 48-72 hours at 25° C.

For the microorganism suspension, microorganisms were used to prepare a 0.5 McFarland suspension in saline. 100 μl of the bacterial suspensions were added to 9.9 ml of Trypticase Soy Broth, and 100 μl of the fungal suspensions were added to 9.9 ml of Saboraud Dextrose Broth.

For the tray preparation, ethanol, methanol, and ethyl acetate extracts of Morinda citrifolia, were used in this study. Morinda citrifolia fruit juice extracts were supplied by Morinda, Inc. Each extract was used to prepare a row of microliter wells. Wells 1 and 6 received 200 μl of extract; wells 2 and 7 received 100 μl of extract; wells 3 and 8 received 50 μl of extract; wells 4 and 9 received 25 μl of extract; wells 5 and 10 received 12.5 μl of extract; and wells 6 and 12 received 6.3 μl of extract. This resulted in each row containing a duplicate series of extract material. Ethanol extracts were placed into rows A-B of a standard microliter tray, methanol extracts were placed into rows C-D of a standard microliter tray, and ethyl acetate extracts were placed into rows E-F of a standard microliter tray. Row G received 200 μl of 95% ethyl alcohol, and Row H received nothing. Trays were then incubated at 37° C. for 48 hours and allowed to dry.

Each microorganism was inoculated into a different tray using the 1/100 suspension of microorganism in media. 100 μls were added to each well. Following inoculation, bacterial isolates were incubated for 24-48 hours at 37° C.. Fungal isolates were incubated for 72 hours at 25° C.. Following incubation, wells were analyzed for growth. A minimal inhibitory concentration (MIC) was determined by noting the lowest concentration of extract that inhibited growth. Results were reported as microliters of extract in the well exhibiting the MIC. Rows G and H served as extract and growth controls.

Several problems had to be overcome in developing this assay. Perhaps the most difficult, was perfecting a method of drying the compounds in such a fashion as to allow them to be resolubilized after they were inoculated. A review of the history of the development of antimicrobials indicates that early experiments in which extracts of penicillin were dried resulted in the total loss of activity. This problem was solved by using low heat for an extended period of time.

The following Tables illustrate the discovered activity. Activity is reported as the smallest volume of dried extract capable of inhibiting growth.

TABLE 1
Activity of Ethanol Extracts

| | | |
|---|---|---|
| E. Coli | 50 | μl |
| S. aureus | 12.5 | μl |
| T. mentagrophytes | ≦6.3-25 | μl |
| A. niger | 100-200 | μl |
| C. albicans | 100 | μl |

TABLE 2
Activity of Methanol Extracts

| | | |
|---|---|---|
| E. Coli | 25-50 | μl |
| S. aureus | ≦6.3 | μl |
| T. mentagrophytes | ≦6.3-12.5 | μl |
| A. niger | 200 | μl |
| C. albicans | 50-100 | μl |

TABLE 3
Activity of Ethyl Acetate Extracts

| | | |
|---|---|---|
| E. Coli | 200->200 | μl |
| S. aureus | 50-200 | μl |
| T. mentagrophytes | 50-100 | μl |
| A. niger | >200 | μl |
| C. albicans | >200 | μl |

TABLE 4
Extracts Tested with E. Coli

| | | | | |
|---|---|---|---|---|
| Ethanol | 50 | 50 | 50 | 50 |
| Methanol | 25 | 50 | 25 | 25 |
| Ethyl Acetate | >200 | >200 | 200 | >200 |

TABLE 5
Extracts Tested with S. Aureus

| | | | | |
|---|---|---|---|---|
| Ethanol | 12.5 | 12.5 | 12.5 | 12.5 |
| Methanol | ≦6.3 | ≦6.3 | ≦6.3 | ≦6.3 |
| Ethyl acetate | 50 | 50 | 200 | 200 |

TABLE 6
Extracts Tested with T. Mentagrophytes

| | | | | |
|---|---|---|---|---|
| Ethanol | ≦6.3 | 25 | ≦6.3 | 25 |
| Methanol | ≦6.3 | 12.5 | ≦6.3 | 12.5 |
| Ethyl Acetate | 50 | 50 | 100 | 100 |

TABLE 7
Extracts Tested with A. Niger

| | | | | |
|---|---|---|---|---|
| Ethanol | 200 | 200 | 100 | 100 |
| Methanol | 200 | 200 | 200 | 200 |
| Ethyl Acetate | >200 | >200 | >200 | >200 |

TABLE 8
Extracts Tested with C. Albicans

| | | | | |
|---|---|---|---|---|
| Ethanol | 100 | 100 | 100 | 100 |
| Methanol | 100 | 100 | 50 | 50 |
| Ethyl Acetate | >200 | >200 | >200 | >200 |

The results of the test showed that activity of Ethanol extracts ranged from ≦6.3 μl to 200 μl; the activity of Methanol extracts ranged from ≦6.3 μl to 200 μl; the activity of Ethyl Acetate extracts ranged from 50 ul to 200 μl; and that ethanol and methanol extracts were the most effective against all of the microorganisms tested.

This study attempts to take the first steps at isolating new antimicrobial compounds from a raw material. This “top down” approach utilized crude extracts of Morinda citrifolia. Results indicated that the ethanol and methanol had activity against all of the microorganisms tested, which further indicated the antifungal activity of Morinda citrifolia.

With the demonstration of antimicrobial activity, it can be said that there exists at least one and possibly several compounds within Morinda citrifolia that are responsible for the antimicrobial activity exhibited herein. As such, other tests and experiments will become necessary to specifically identify and isolate these. Most likely, future research will involve purifying the extracts discussed herein using standard separation techniques, which will involve defining some of the myriad of compounds that are present in these extracts. Once isolated, each can be tested for antimicrobial activity.

### EXAMPLE TWO

The purpose of this experiment was to determine the mean inhibitory concentration (MIC) of selected Morinda citrifolia fruit juice extracts against three common pathogenic fungi and two common bacteria.

The organism used were Aspergillus niger (ATCC 6275); Candida albicans (ATCC 10231); Trichophyton mentagrophytes (ATCC 9533); Staphlococcus aureus (ATCC 29213); and Escherichia coli (ATCC 9533).

For the Morinda citrifolia fruit juice extracts, ethanol, methanol, ethyl acetate, and aqueous extracts of were prepared using the appropriate solvents.

The sterile media preparations (1 liter) included: for fungi, a Sabouraud Dextrose Broth (SDB); for bacteria, a Mueller Hinton Broth (MHB); autoclave at 121° C. for 20 minutes.

The organism suspension preparations included plating each organism on appropriate media, incubate and confirm identity, prepare a 0.5 McFarland suspension of each organism, and add 0.1 ml of the organism to 9.9 ml of the appropriate media (SDB or MHB).

To prepare the Morinda citrifolia juice extracts, using the appropriate media, the extracts were dried and then diluted to a final concentration of 2 mg/ml. The extracts were then stored in −20° C. freezers until ready for fungal plating. These 2 mg/ml final volumes were used as Morinda citrifolia stock solutions.

Thirteen test tubes were labeled as follows in table 9:

TABLE 9
Test Tube Labels

1/1
1/2
1/4
1/8
1/16
1/32
1/64
1/128
1/256
1/512
1/1024
Growth control
Non-inoculated control

100 μl of Morinda citrifolia stock solution was added to Tube 1/1 and 100 μl to Tube ½. 100 μl of sterile media was added to Tubes: ½, ¼, ⅛, 1/16, 1/32, 1/64, 1/128, 1/256, 1/512, 1/1024, Growth control, and Non-inoculated control.

Tube ½ was mixed well and 100 μl removed and added to Tube ¼. This two-fold dilution procedure was continued for Tubes ⅛, 1/16, 1/32, 1/64, 1/128, 1/256, 1/512, and 1/1024. Discard 100 μl from Tube 1/1024. No diluted Morinda citrifolia solutions were added to Tubes GC or NC. These were the control tubes. At this point all tubes contained 100 μl.

Because we know that we started with 2 mg/ml (i.e. 2000 μg/ml) of extract stock solution, the serial two fold dilution resulted in the following concentrations of Morinda citrifolia fruit juice extract as shown in the table 10 below.

TABLE 10
Serial Dilution

| Tube # | Dilution | Concentration of Extract |
|---|---|---|
| 1 | 1/1 | 2000 μg/ml |
| 2 | 1/2 | 1000 μg/ml |
| 3 | 1/4 | 500 μg/ml |
| 4 | 1/8 | 250 μg/ml |
| 5 | 1/16 | 125 μg/ml |
| 6 | 1/32 | 62.50 μg/ml |
| 7 | 1/64 | 31.25 μg/ml |
| 8 | 1/128 | 15.13 μg/ml |
| 9 | 1/256 | 7.56 μg/ml |
| 10 | 1/512 | 3.78 μg/ml |
| 11 | 1/1024 | 1.89 μg/ml |
| 12 | GC | No extract |
| 13 | NC | No organism |

During inoculation, 100 μl of organism suspension were added to all of the tubes except Tube Non-inoculated control (NC). 100 μl of additional media was added to NC. All tubes were incubated at the appropriate temperatures and intervals—for fungi, 25° C. for 5-7 days; for bacteria, 37° C. for 24-48 hours.

the results were recorded by observing turbidity. The presence of turbidity indicated growth, while the absence of turbidity indicated inhibition of growth. For any extract, a result was valid only if there was turbidity (i.e. growth) in the Tube Growth control, and no turbidity in the Tube Non-inoculated control (i.e. no growth). The MIC was determined as the last tube in the series (i.e. the most diluted tube) with no turbidity.

the following, table 11, represents the mean inhibitory concentration (μg/ml):

TABLE 11
Mean Inhibitory Concentration

| | EtOH | MeOH | EtAc |
|---|---|---|---|
| C. albicans | 1000 | 250-1000 | >2000 |
| A. niger | 1000-2000 | 1000-2000 | >2000 |
| T. mentagr. | ≦7.56 | ≦7.56 | 250-1000 |
| S. aureus | 31.25-62.50 | 31.25-62.50 | 1000-2000 |
| E. coli | 250 | 62.50-250 | >2000 |

Results indicate that the ethanol and methanol Morinda citrifolia extracts had meaningful activity against all of the microorganisms tested. Preliminary drying studies indicated that the activity using the ethanol and methanol extracts was in the 5-10 mg/ml range. Ethyl acetate extracts contained <10% of the amount found in the ethanol and methanol extracts.

From this initial phase of the study, it can clearly be established that Morinda citrifolia fruit juice or the extracts thereof exhibit a substantial amount of antifungal activity. However, each extract contains hundreds of compounds. Indeed, at 1000 μ/ml, there may be 100 compounds at concentrations of 10 μl/ml each. Thus, since the extracts tested were not purified antimicrobial compounds, even very high MIC's may be meaningful. Later tests described below set forth some specific compounds that were fractioned or extracted out of Morinda citrifolia fruit juice concentrate.

### EXAMPLE THREE

For the following experiment, the minimum inhibitory concentration (MIC) of an antibacterial is defined as the maximum dilution of the product that will still inhibit the growth of a test microorganism. The minimum lethal concentration (MLC) of an antibacterial is defined as the maximum dilution of the product that killed a test organism. MIC/MLC values can be determined by a number of standard test procedures. The most commonly employed methods are the tube dilution method and agar dilution methods. The tube dilution method was proposed for this product to determine the MIC, and plating aliquots from dilutions demonstrating possible inhibition of growth to determine the MLC. Serial dilutions were made of the products in bacterial growth media. The test organisms were added to the dilutions of the products, incubated, and scored for growth. All tests were performed in triplicate.

This procedure is a standard assay for antimicrobials. The procedure incorporates the content and intent of the American Society for Microbiology (ASM) recommended methodology. The tube dilution method employs dilutions of the test product in a bacterial growth media, inoculation with a predetermined test organism concentration, and visualization of growth after incubation. Tube dilution procedures are limited to products which do not precipitate or cloud the growth media within the expected endpoint range.

For the culture preparation procedure, the test organisms used were Escherichia coli 0157H7 ATCC #43888; Staphylococcus aureus ATCC #6538; Bacillus subtilis ATCC #19659; Salmonella choleraesuis serotype enteritidis ATCC #13706; Listeria monocytogenes ATCC #19111; Candida albicans ATCC #10231; and Streptococcus mutans ATCC #25175.

From stock, the test organisms were transferred to soybean casein digest broth (SCDB) and incubated at 37±2° C. for 24-48 hours for bacteria, and 20-25° C. for yeast. If needed, the suspensions were adjusted to approximately 10⁸ colony forming units (CFU) per mL, by visual turbidity, in physiological saline solution (PHSS) and a standard plate count was performed to determine starting titers. The yeast culture was plated onto Sabouraud dextrose agar (SDEX) and incubated at 20-25° C. for 2-4 days, S. mutans was incubated at 37±2° C. for 3-5 days, and all other bacteria were incubated at 37±2° C. for 18-24 hours.

For the Mean Inhibitory Concentration (MIC) test procedure, the test product was adjusted to a neutral pH for the purpose of this test. The pH was recorded before and after adjustments had been made. Each test product was diluted 1:2 serially in sterile water. Dilutions were selected that would show the MIC/MLC endpoint. Each test product evaluation was performed in triplicate for each organism. The product dilutions were added to an equal volume of 2×SCDS to provide an additional 1:2 dilution. Three positive control tubes were prepared for each test organism by mixing sterile water with equal volumes of 2×SCDB. Three negative control tubes were prepared by mixing the highest dilution tested of the test product with equal volumes of 2×SCDB. No test organisms were added to these tubes. Three media control tubes were prepared by mixing sterile water with equal volumes of 2×SCDB. No test organisms were added to these tubes either.

Approximately 0.05 mL of each test organism suspension was added to the sample and positive control tubes. The bacteria test tubes were incubated at 37±2° C. for 18-24 hours and yeast test tubes were incubated at 20-25° C. for 2-4 days. After incubation, growth was scored as negative (0) or positive (+) for each tube.

For the Mean Lethal Concentration (MLC) test procedure, only tubes suspected of not having any growth were tested. A 1.0 mL aliquot was removed from each tube and serial 1/10 dilutions were made in neutralizer broth up to 1/1000. An aliquot of each dilution was plated on neutralizer agar (NUAG). For a positive control, 10-100 CFU were plated onto NUAG. A negative control was made by plating 2×SCDB onto NUAG. The plates were incubated 20-25° C. for 2-4 days for yeast, and 37±2° C. for 18-24 hours for all bacteria except for S. mutans.

With regards to what is known as neutralization verification, the lowest dilution of the test product tested for MLC was tested for neutralization recovery for each test organism. In triplicate, 0.5 mL aliquots of the most concentrated test product were plated on NUAG. The plates were spiked with 10-100 CFU of each test organism. For comparison, three plates of NUAG without the test product were also spiked with the same 10-100 CFU for each of the test organisms.

With the exception of S. mutans, all organisms were inhibited by neutralized Morinda citrifolia concentrate at a 1:2 concentration. None of the dilutions tested were able to demonstrate lethality for any of the organisms. Neither inhibition nor lethality was demonstrated by the neutralized Morinda citrifolia concentrate when tested against S. mutans.

The MIC results for all organisms are summarized in Tables 12-18. The MLC results for each organism are summarized in Tables 19-25. Since S. mutans did not have any dilutions that were scored as having no growth for the MIC portion of the test, MLC was not performed for this organism.

The neutralization recoveries for all test organisms ranged from 40-97%. The neutralization recovery of the neutralizing media used in the study is summarized in Table 25.

TABLE 12
Mean Inhibitory Concentration Results for
Escherichia coli O157H7 ATCC #43885

| DILUTION | GROWTH +/0 | | |
|---|---|---|---|
| 1:2 | 0 | 0 | 0 |
| 1:4 | + | + | + |
| 1:8 | + | + | + |
| 1:16 | + | + | + |
| 1:32 | + | + | + |
| 1:64 | + | + | + |
| Positive | + | + | + |
| Negative | 0 | 0 | 0 |
| Media | 0 | 0 | 0 |

Titer: 7.0 × 10⁸ CFU/mL
Inoculating volume = 0.05 mL

TABLE 13
Mean Inhibitory Concentration Results for
Staphylococcus aureus ATCC #6538

| DILUTION | GROWTH +/0 | | |
|---|---|---|---|
| 1:2 | 0 | 0 | 0 |
| 1:4 | + | + | + |
| 1:8 | + | + | + |
| 1:16 | + | + | + |
| 1:32 | + | + | + |
| 1:64 | + | + | + |
| Positive | + | + | + |
| Negative | 0 | 0 | 0 |
| Media | 0 | 0 | 0 |

Titer: 6.5 × 10⁸ CFU/mL
Inoculating volume = 0.05 mL

TABLE 14
Mean Inhibitory Concentration Results for Bacillus
subtilis ATCC #19659

| DILUTION | GROWTH +/0 | | |
|---|---|---|---|
| 1:2 | 0 | 0 | 0 |
| 1:4 | + | + | + |
| 1:8 | + | + | + |
| 1:16 | + | + | + |
| 1:32 | + | + | + |
| 1:64 | + | + | + |
| Positive | + | + | + |
| Negative | 0 | 0 | 0 |
| Media | 0 | 0 | 0 |

Titer: 8.5 × 10⁷ CFU/mL
Inoculating volume = 0.05 mL

TABLE 15
Mean Inhibitory Concentration Results for
Salmonella choleraesuis serotype enteritidis ATCC #13706

| DILUTION | GROWTH +/0 | | |
|---|---|---|---|
| 1:2 | 0 | 0 | 0 |
| 1:4 | + | + | + |
| 1:8 | + | + | + |
| 1:16 | + | + | + |
| 1:32 | + | + | + |
| Positive | + | + | + |
| Negative | 0 | 0 | 0 |
| Media | 0 | 0 | 0 |

Titer: 4.8 × 10⁸ CFU/mL
Inoculating volume = 0.05 mL

TABLE 16
Mean Inhibitory Concentration Results for Listeria
monocytogenes ATCC #19111

| DILUTION | GROWTH +/0 | | |
|---|---|---|---|
| 1:2 | 0 | 0 | 0 |
| 1:4 | + | + | + |
| 1:8 | + | + | + |
| 1:16 | + | + | + |
| 1:32 | + | + | + |
| 1:64 | + | + | + |
| Positive | + | + | + |
| Negative | 0 | 0 | 0 |
| Media | 0 | 0 | 0 |

Titer: 3.9 × 10⁸ CFU/mL
Inoculating volume = 0.05 mL

TABLE 17
Mean Inhibitory Concentration Results for Candida
albicans ATCC #10231

| DILUTION | GROWTH +/0 | | |
|---|---|---|---|
| 1:2 | 0 | 0 | 0 |
| 1:4 | + | + | + |
| 1:8 | + | + | + |
| 1:16 | + | + | + |
| 1:32 | + | + | + |
| 1:64 | + | + | + |
| Positive | + | + | + |
| Negative | 0 | 0 | 0 |
| Media | 0 | 0 | 0 |

Titer: 1.3 × 10⁸ CFU/mL
Inoculating volume = 0.05 mL

TABLE 18
Mean Inhibitory Concentration Results for
Streptococcus mutans ATCC #25175

| DILUTION | GROWTH +/0 | | |
|---|---|---|---|
| 1:2 | + | + | + |
| 1:4 | + | + | + |
| 1:8 | + | + | + |
| Positive | + | + | + |
| Negative | 0 | 0 | 0 |
| Media | 0 | 0 | 0 |

Titer: 1.0 × 10⁷ CFU/mL
Inoculating volume = 0.05 mL

TABLE 19
Mean Lethal Concentration Results for Escherichia
coli 0157H7 ATCC #43588

| | | DILUTION | | | |
|---|---|---|---|---|---|
| DILUTION | REPLICATE | 10⁰ | 10⁻¹ | 10⁻² | 10⁻³ |
| 1:2 | 1 | TNTC | TNTC | TNTC | 245 |
| | 2 | TNTC | TNTC | TNTC | 239 |
| | 3 | TNTC | TNTC | TNTC | 215 |

Volume plated = 0.5 mL
TNTC = Too Numerous To Count

TABLE 20
Mean Lethal Concentration Results for
Staphylococcus aureus ATCC #6538

| | | DILUTION | | | |
|---|---|---|---|---|---|
| DILUTION | REPLICATE | 10⁰ | 10⁻¹ | 10⁻² | 10⁻³ |
| 1:2 | 1 | TNTC | TNTC | TNTC | 200 |
| | 2 | TNTC | TNTC | TNTC | 134 |
| | 3 | TNTC | TNTC | TNTC | 114 |

Volume plated = 0.5 mL
TNTC = Too Numerous To Count

TABLE 21
Mean Lethal Concentration Results for Bacillus
subtilis ATCC #19659

| | | DILUTION | | | |
|---|---|---|---|---|---|
| DILUTION | REPLICATE | 10⁰ | 10⁻¹ | 10⁻² | 10⁻³ |
| 1:2 | 1 | 27 | 3 | 0 | 0 |
| | 2 | 25 | 2 | 0 | 0 |
| | 3 | 18 | 2 | 0 | 0 |

Volume plated = 0.5 mL

TABLE 22
Mean Lethal Concentration Results for Salmonella
choleraesuis serotype enteritidis ATCC #13706

| | | DILUTION | | | |
|---|---|---|---|---|---|
| DILUTION | REPLICATE | 10⁰ | 10⁻¹ | 10⁻² | 10⁻³ |
| 1:2 | 1 | TNTC | TNTC | 41 | 7 |
| | 2 | TNTC | TNTC | 75 | 5 |
| | 3 | TNTC | TNTC | 63 | 6 |

Volume plated = 0.5 mL
TNTC = Too Numerous To Count

TABLE 23
Mean Lethal Concentration Results for Listeria
monocytogenes ATCC #19111

| | | DILUTION | | | |
|---|---|---|---|---|---|
| DILUTION | REPLICATE | 10⁰ | 10⁻¹ | 10⁻² | 10⁻³ |
| 1:2 | 1 | TNTC | TNTC | TNTC | 109 |
| | 2 | TNTC | TNTC | TNTC | 109 |
| | 3 | TNTC | TNTC | TNTC | 179 |

Volume plated = 0.5 mL
TNTC = Too Numerous To Count

TABLE 24
Mean Lethal Concentration Results for Candida
albicans ATCC #10231

| | | DILUTION | | | |
|---|---|---|---|---|---|
| DILUTION | REPLICATE | 10⁰ | 10⁻¹ | 10⁻² | 10⁻³ |
| 1:2 | 1 | TNTC | TNTC | TNTC | 168 |
| | 2 | TNTC | TNTC | TNTC | 117 |
| | 3 | TNTC | TNTC | TNTC | 138 |

Note:
Volume plated = 0.5 mL
TNTC = Too Numerous To Count

TABLE 25
Neutralization

| | POSITIVE | | | | NEUTRALIZA- | | | | |
|---|---|---|---|---|---|---|---|---|---|
| | COUNT | | | | TION COUNT | | | | PERCENT |
| ORGANISM | 1 | 2 | 3 | AVE | 1 | 2 | 3 | AVE | RECOVERY |
| E. coli 0157H7 | 60 | 63 | 58 | 60 | 53 | 50 | 73 | 59 | 97% |
| S aureus | 48 | 65 | 38 | 50 | 49 | 44 | 42 | 45 | 89% |
| B. subtilis | 53 | 61 | 53 | 56 | 25 | 20 | 22 | 22 | 40% |
| S. choleraesuis | 38 | 43 | 36 | 39 | 34 | 34 | 31 | 33 | 85% |
| L. | 43 | 38 | 22 | 34 | 26 | 31 | 34 | 30 | 88% |
| monocytogenes | | | | | | | | | |
| C. albicans | 36 | 25 | 21 | 27 | 20 | 12 | 27 | 20 | 72% |
| S. mutans | 11 | 19 | 13 | 14 | 9 | 16 | 14 | 13 | 91% |

### EXAMPLE FOUR

Experiments were done to identify the one or more specific compounds or fractions existing within the several Morinda citrifolia product(s) that is/are responsible for effectuating antifungal activity within the body once introduced therein.

Morinda citrifolia fruit juice was fractioned to obtain Morinda citrifolia n-hexane fractions, Morinda citrifolia CL₂CL₂, Morinda citrifolia ETOAc fractions, and Morinda citrifolia BuOH fractions, each of a specific concentration. Each of these were studied to determine their antimicrobial activity using the Aspergillus niger (ATCC 6275); Candida albicans (ATCC 10231); Staphlococcus aureus (ATCC 29213); and Escherichia coli(ATCC 9533) organisms. Other Morinda citrifolia products may also be fractioned in a similar manner as described herein.

In preparation, each extract was tested by preparing a series of concentrations in a microtiter tray. The first well of each series received 200 μl, the second 100 μl, the third 50 μl, the fourth 25 ul, the fifth 12.5 μl, and the sixth 6.3 μl. Trays were incubated at 35-37° C. for 72 hours. At this time all of the extracts had dried.

For the preparation of the organisms, ATCC isolate was plated on an appropriate media, and incubated. Following incubation, a 0.5 McFarland suspension of the organism was prepared in saline. 100 μl of this suspension was added to 9.9 ml of the appropriate media. 200 μl of the organism suspension were added to each well of the series, and used to suspend test material. An empty well was inoculated to serve as a growth control, and one well was inoculated with media to serve as a negative control. Trays were incubated at the appropriate temperatures, for the appropriate intervals. (For the bacterial samples this was 35±2° C. for 24-48 hours. For fungi this was 20-25° C. for 5-7 days).

The growth control well was observed for the presence of turbidity, and the negative control was observed for the absence of turbidity. A result was only valid, if there was growth in the Growth Control well, and no growth in the non-inoculated well. Following this, each of the other wells were observed for the presence of turbidity. Results were recorded. The trays were then placed on a Multiskan Plate reader. Absorbance at 550 nm was recorded.

The minimum inhibitory concentration (MIC) was the last tube in the series, which was not turbid. The results of the test are presented below in the following tables, where activity is reported as mg/ml.

TABLE 26
Activity of Morinda citrifolia fruit juice concentrate

| | |
|---|---|
| E. Coli | 25 mg |
| S. aureus | 25 mg |
| A. niger | >50 mg |
| C. albicans | 50 mg |

TABLE 27
Activity of Morinda citrifolia hexane fraction

| | |
|---|---|
| E. Coli | 25 mg |
| S. aureus | 25 mg |
| A. niger | 25 mg |
| C. albicans | 12.5 mg |

TABLE 28
Activity of Morinda citrifolia ETOAc fraction

| | |
|---|---|
| E. Coli | 6.3 mg |
| S. aureus | 3.1 mg |
| A. niger | 25 mg |
| C. albicans | 12.5 mg |

TABLE 29
Activity of Morinda citrifolia n-BuOH fraction

| | |
|---|---|
| E. Coli | >12.5 mg |
| S. aureus | 25 mg |
| A. niger | >50 mg |
| C. albicans | >50 mg |

Morinda citrifolia fractions and extracts exhibited inhibitory and preventative activity against the organisms being tested.

Two problems were encountered in this study. The first is that there was a problem getting some of the higher concentrations of the ETOAc fractions or extracts into solution. As a result when these were read, precipitation was observed. This precipitation did not interfere with the visual readings, but did interfere with the absorbance measurements. A second problem is that the n-hexane fractions or extracts appeared to etch the plastic in the microtiter plate. This too caused problems with the absorbance, but not the visual readings. Additionally, due to a lack of supplied compounds, the fourth tray did not have sufficient n BuOH to prepare all of the concentrations. As a result the E. coli result is reported as >12.5 mg/ml.

### EXAMPLE FIVE

Experiments were conducted to verify that Morinda citrifolia products can inhibit the growth of fungi, and to verify that Morinda citrifolia products could be used as a post-harvest spray. In one set of qualitative experiments processed Morinda citrifolia product was sprayed onto strawberry plants. The Morinda citrifolia sprayed strawberries kept fresh longer than control group. Additionally, the yield of Morinda citrifolia sprayed was larger than control. Morinda citrifolia sprayed strawberries were sweeter (higher brix) than control. Plants have the immune-like system called intacellular pathogenesis (IP). IP provides a basis for allowing health plants to resistant pathogens. The present invention contemplates the possibility that chemicals present in the processed Morinda citrifolia activate the IP pathway.

### EXAMPLE SIX

In another experiment harvested strawberries were sprayed with Morinda citrifolia products. Four groups of strawberries were treated. Groups one through three were sprayed with a serial dilution of processed Morinda citrifolia (Group 1=undiluted, Group 2 was diluted 1:200 and Group 3 was diluted 1:1000). Group 4 was sprayed with Benlate, which had been diluted 1:500. Benlate is the artificial pesticide certified by the Department of Agriculture in Japan. The strawberries were observed for four days. Qualitative analysis indicated that mold infections were prevented on strawberries, which had been sprayed with processed Morinda citrifolia.

### EXAMPLE SEVEN

In another experiment a strawberry farmer whose strawberries were suffering from powdery mildew caused by Sphaerotheca spp. sprayed processed Morinda citrifolia (diluted 1:400 with water) on the strawberries. The fungal infections decreased. The strawberry became thicker and sweeter than usual. The present invention contemplates the possibility that the processed Morinda citrifolia kill bacteria and fungi directly and/or enhances the immune system of plants. Further, it is contemplated by the present invention that the enhanced immune system of plants is affected by the application of processed Morinda citrifolia to the extent that the application supplies nutrients and balances the normal flora of the soil.

### EXAMPLE EIGHT

In another experiment, four planters were installed in a green house. Ten seedlings of strawberries (Fragaria ananassa: Tochiotome variety) were planted each planter. The top left planter was sprayed with processed Morinda citrifolia (Group 1). The top right planter was a control group (Group 2). The bottom left planter was a control group(Group3). The bottom right planter was sprayed with processed Morinda citrifolia (Group 4). For six months the plants were merely watered, no processed Morinda citrifolia or fungi was sprayed. Each planter was watered with 500 ml of water every 4 days. The flowers were removed once visible.

For one month following the six month watering period the plants were sprayed with fungi in addition to the above prescribed watering regime. The strawberry leaves were infected by fungi, Sphaerotheca humitli burrill. The fungi was pounded and diluted into 450 ml distilled water, and 100 ml water was sprayed into all groups. The spray of the fungi was conducted every four days.

Beginning in the ninth month of the experiment 1 ml of processed Morinda citrifolia juice was diluted into 199 ml of the distilled water, and the solution was sprayed on Groups 1 and 4. 200 of distilled water without processed Morinda citrifolia was sprayed to each control group (Groups 2 and 3). The processed Morinda citrifolia spray was sprayed every four days. The experiment is still being conducted but results similar to those described above are expected.

### EXAMPLE NINE

Morinda citrifolia juice was used in an experiment conducted in a strawberry green house. There were six furrows of length 30 m with 80 Tochiotome strawberry plants planted on each furrow. Each furrow was divided into two equal sections, with diluted Morinda citrifolia juice dispersed on one side while the same amount of water is dispersed on the other section, which was used as control.

Morinda citrifolia juice was diluted with water and each time, three liter of the solution per one sq. m was dispersed on the strawberry plants. Dispersion began 12 days prior to formation of strawberry fruits, once every two days for total of five dispersions. In the first three dispersions, Morinda citrifolia juice was diluted 200 mass-times with water, and the was diluted 300 mass-times for the last two dispersions. After harvesting of strawberries, amount of yield, sugar content and freshness maintenance were examined for the control group and Morinda citrifolia juice dispersed group.

Only the strawberries measuring longer than 3.0 cm from the calyx to the tip of the fruit were included to determine, using a scale, the amount of harvest in weight. The yield was 600 gram (38 strawberries) for the control group, while that for the group on which Morinda citrifolia juice was dispersed was 1400 gram (96 strawberries). From the comparison, it may be concluded that coating and dispersion of Morinda citrifolia juice speeds up growth of the strawberries, reaching harvest criteria of 3 cm faster. Moreover, during experiment white flour disease were seen on some plants, but dispersion of Morinda citrifolia prevent the spread of the disease.

Sugar content was measured with a digital sugar meter (measurement accuracy of ±0.2 BRIX) made by Kyoto Denshi Kogyo KK. After removing calyx, 10 strawberries were placed in a blender and thoroughly agitated. Resulting strawberry juice was poured into the sugar meter and the total five measurements were made, from which a mean value was determined. The mean value of sugar content for the group with Morinda citrifolia dispersion was 8.0 Brix while that of the control group was 7.1 Brix. From the experiment, it was found that sugar content of the strawberry increased 13% with dispersion of Morinda citrifolia juice.

Next, in order to examine the maintenance of freshness after harvest, strawberries harvested were kept and observed for ten days in a refrigerator. Some of the fruits in the control group were found to be rotten with white mold at 10 days after harvest, while no mold was found and surface was tight for the strawberries from the Morinda citrifolia group. From this, it was concluded that dispersion of Morinda citrifolia juice on the plant extends freshness period of the strawberry and prevents mold growth.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

### EXAMPLE 10

Morinda citrifolia products processed according to this invention have been utilized to promote lawn care. In various cases, processed Morinda citrifolia products have been applied to lawns. The application of processed Morinda citrifolia ameliorated fungal infection on lawns. The fungal infections had a phenotype of causing the lawn to turn a brown color. Further, the application of Morinda citrifolia prevented further recurrence of fungal infections on lawns to which it was applied.

1. A formulation for inhibiting fungal and microbial growth on plants comprising:
at least one processed Morinda citrifolia product present in an amount between about 0.01 and 99.99 percent by weight.

2. A formulation as is claim 1, wherein said formulation is comprised of an extract or mixture of extracts selected from a list consisting of fruit, stem, seed, pericarp, root bark, leaves and root of Morinda citrifolia.

3. A formulation as in claim 2, wherein extract or mixture of extracts selected from a list consisting of fruit, stem, seed, pericarp, root bark, leaves and root of Morinda citrifolia are diluted by a factor of 1-10,000 times (in weight) with water.

4. A formulation as is claim 1, wherein said formulation is made into liquid, granule, powder or paste agent with appropriate carrier materials.

5. A formulation as is claim 1, wherein the formulation is dissolved or dispersed in water.

6. A formulation as is claim 5, wherein the Morinda citrifolia product is diluted by a factor of 1-10,000 times by weight with water.

7. A formulation as is claim 1, herein the formulation is further comprised of at least one fertilizer component.

8. A formulation as is claim 1, wherein said fertilizer component is selected from a list comprised of ammonium sulfate, urea, potassium, nitrogen and ammonium chloride, chicken manure, cow manure, guano, worm castings, insect manure, saw dust, rice bran, garlic oil, fish oil, vermiculite, montmorillonite, active carbon, charcoal, diatomite, talc, alfalfa meal and pellets, nitrogen, phosphorus, potassium, dried shredded remains of sugar beets, corn gluten, cottonseed meal, extracts or pulverized parts of several kelp or algae, soybean meal, animal processing by-products, blood meal, bonemeal, compost or fish byproducts.

9. The formulation of claim 1, wherein said processed formulation further comprises Quercetin.

10. The formulation of claim 9, further comprising Rutin as an additional active ingredient that synergistically works with said Quercetin to inhibit said fungal and microbial growth.

11. The method of claim 10, wherein said Rutin is present in an amount between about 0.1 and 10 percent by weight.

12. A formulation for inhibiting fungal and microbial growth on plants, said formulation comprising between 0.01 and 10% by weight of Morida citrifolia n-hexane fraction.

13. The formulation of claim 12, wherein said Morinda citrifolia fraction comprises a Morinda citrifolia CL₂CL₂ fraction.

14. The formulation of claim 12, wherein said Morinda citrifolia fraction comprises a Morinda citrifolia ETOAc fraction.

15. The formulation of claim 12, wherein said Morinda citrifolia fraction comprises a Morinda citrifolia an n-BuOH fraction.

16. A formulation as is claim 12, wherein said formulation is comprised of an extract or mixture of extracts selected from a list consisting of fruit, stem, seed, pericarp, root bark, leaves and root of Morinda citrifolia.

17. A formulation as in claim 12, wherein the formulation is diluted by a factor of 1-10,000 times by weight prior or during application.

18. A formulation as is claim 12, wherein said formulation is made into liquid, granule, powder or paste agent with appropriate carrier materials.

19. A formulation as is claim 12, wherein the formulation is dissolved or dispersed in water.

20. A formulation as is claim 12, herein the formulation is further comprised of at least one fertilizer component.

21. A formulation as is claim 20, wherein said fertilizer component is selected from a list comprised of ammonium sulfate, urea, potassium, nitrogen and ammonium chloride, chicken manure, cow manure, guano, worm castings, insect manure, saw dust, rice bran, garlic oil, fish oil, vermiculite, montmorillonite, active carbon, charcoal, diatomite, talc, alfalfa meal and pellets, nitrogen, phosphorus, potassium, dried shredded remains of sugar beets, corn gluten, cottonseed meal, extracts or pulverized parts of several kelp or algae, soybean meal, animal processing by-products, blood meal, bonemeal, compost or fish byproducts.

22. The formulation of claim 21, wherein said processed formulation further comprises Quercetin.

23. The formulation of claim 23, further comprising Rutin as an additional active ingredient that synergistically works with said Quercetin to inhibit said fungal and microbial growth.

24. The method of claim 23, wherein said Rutin is present in an amount between about 0.1 and 10 percent by weight.

25. A method for inhibiting fungal and microbial activity on plants, said method comprising the steps of:
exposing said plant to a formulation, said formulation comprising:
a processed Morinda citrifolia product present in an amount by weight between about 0.01-99.99 percent.

26. A method as in claim 25, wherein said plant is repeatedly exposed until all harmful fungi and microbials and related effects are ameliorated.

27. A method as in claim 25, wherein said formulation is further comprised of at least one active ingredient.

28. The method of claim 27, wherein the active ingredient is selected from a list comprised of Quercetin, Rutin, n-hexane extract, CL₂CL₂ extract, ETOAc extract, and n-BuOH extract.

29. The method of claim 25, wherein the method further comprises the step of exposing plant material selected from a list consisting of: fruits, vegetables, leafy vegetables, root vegetables, grains, flower and bulbs.

30. The method of claim 25, further comprising the step of exposing the plant to the formulation in at least one of the following ways: the formulation may be sprayed or irrigated in the soil prior to planting; the formulation may be sprayed or irrigated in the soil during plant growth; coating the plant during cutting dividing or re-planting the plant; coating seed or bulb during planting; coating wilting flowers and shrubs; dispersing on water grown plant; coating plants infected with bacteria or virus; coating cut flowers after harvest; or coating crop and flower after harvest.

31. A formulation as is claim 25, wherein said formulation is comprised of an extract or mixture of extracts selected from a list consisting of fruit, stem, seed, pericarp, root bark, leaves and root of Morinda citrifolia.

32. A formulation as in claim 31, wherein extract or mixture of extracts selected from a list consisting of fruit, stem, seed, pericarp, root bark, leaves and root of Morinda citrifolia are diluted by a factor of 1-10,000 times (in weight) with water.

33. A formulation as is claim 25, wherein said formulation is made into liquid, granule, powder or paste agent with appropriate carrier materials.

34. A formulation as is claim 25, wherein the formulation is dissolved or dispersed in water.

35. A formulation as is claim 25, wherein the Morinda citrifolia product is diluted by a factor of 1-10,000 times by weight with water.

36. A formulation as is claim 25, herein the formulation is further comprised of at least one fertilizer component.

37. A formulation as is claim 36, wherein said fertilizer component is selected from a list comprised of ammonium sulfate, urea, potassium, nitrogen and ammonium chloride, chicken manure, cow manure, guano, worm castings, insect manure, saw dust, rice bran, garlic oil, fish oil, vermiculite, montmorillonite, active carbon, charcoal, diatomite, talc, alfalfa meal and pellets, nitrogen, phosphorus, potassium, dried shredded remains of sugar beets, corn gluten, cottonseed meal, extracts or pulverized parts of several kelp or algae, soybean meal, animal processing by-products, blood meal, bonemeal, compost or fish byproducts.

38. A method for inhibiting harmful fungal and microbial activity on plants, said method comprising the steps of:
initially exposing said plant to as formulation comprising at least one extract from Morinda citrifolia present in an amount between about 0.01% and 99.9% by weight; and
regularly repeating the step of exposing said plant to said formulation.

39. The method of claim 38, wherein said processed Morinda citrifolia product is selected from the group consisting of processed Morinda citrifolia fruit juice, processed Morinda citrifolia puree juice, processed Morinda citrifolia dietary fiber, processed Morinda citrifolia oil, processed Morinda citrifolia fruit juice concentrate, processed Morinda citrifolia puree juice concentrate, processed Morinda citrifolia leaves, processed Morinda citrifolia roots, processed Molinda citrifolia root bark, processed Morinda citrifolia stems, processed Morinda citrifolia seeds and processed Morinda citrifolia oil extract.
